(19) United States
(12) Patent Application Publication
(10) Pub. No.: US 2015/0010528 A1
(43) Pub. Date: Jan. 8, 2015

(54) ANESTHETIC COMPOSITION, FORMULATION AND METHOD OF USE

(71) Applicant: Stuart L. WEG, Franklin Lakes, NJ (US)

(72) Inventor: Stuart L. WEG, Franklin Lakes, NJ (US)

(21) Appl. No.: 14/231,211

(22) Filed: Mar. 31, 2014

Related U.S. Application Data
(63) Continuation of application No. 12/291,343, filed on Nov. 6, 2008, now abandoned.
(60) Provisional application No. 60/985,976, filed on Nov. 6, 2007.

(51) Int. Cl. A61K 38/47; A61M 5/19; A61K 31/445; A61M 5/24; A61K 31/167; A61K 31/137

(52) U.S. Cl. CPC A61K 38/47; A61K 31/167; A61K 31/137; A61K 31/445; A61M 5/2448; A61M 5/19; C12Y 302/01035; A61M 2202/048
USPC 424/94.62; 604/82

(57) ABSTRACT

An anesthetic composition for use e.g. in the administration of a local anesthetic by injection comprises a first component, which comprises hyaluronidase, and a second component which comprises an anesthetic preparation. The composition is both effective and highly shelf stable, and has as an advantage that it may be stored and administered at room temperature. In a particular embodiment, the hyaluronidase is prepared in dry powder form, as by lyophilization. The anesthetic component may be selected from a group of known anesthetics, such as lidocaine, polocaine, xylocaine, novocaine, procaine, prilocaine, bupivacaine, mepivacaine, carbocaine, etidocaine and chincocaine. The composition may be prepared in unit dosage forms, including a single dosage form, for a variety of purposes, and such unit dosage forms may be prepared in a plural chambered syringe or like dispenser, whereby the components are not mixed until administration.

### RELATED APPLICATION

The present application claims the benefit under 35 U.S.C. §119 of U.S. Provisional Application No. 60/985,976, filed Nov. 6, 2007, the contents of which is hereby incorporated by reference in its entirety.

### BACKGROUND OF THE INVENTION

1. Field of the Invention

The present invention relates to the area of pharmaceutical chemistry, and more particularly, to formulations and compositions for use in the administration of anesthesia.

2. Description of the Related Art

The use of anesthetic compositions and their administration is longstanding and broad in application. Local anesthesia is utilized when selected procedures, both medical and dental, are involved, where the nature of the procedure only requires that the tissues that are locally adjacent to the procedure need to be desensitized. By contrast, more comprehensive, and correspondingly, invasive procedures will require more general or systemic desensitization by general anesthesia.

In both instances, one of the difficulties attending the administration and achievement of the desensitization by an anesthetic is the speed with which the anesthetic or desensitized state is established. Generally, the anesthetic must surmount the cell walls and achieve ingress for the desensitization to commence, and the time for such effect to be achieved can vary. Likewise, and particularly in the instance where local administration via injection is concerned, the lag time for the anesthetic effect to take hold can be protracted.

A variety of anesthetic compositions and corresponding methods of administration are known, however, this last mentioned problem has yet to be significantly overcome. In addition, and in the instance where injectable dosage forms are concerned, the anesthesiologist ideally wishes to have such dosage forms pre-measured and prepared for immediate use. Owing to the limited shelf life of some anesthetic compositions, however, such dosage forms must either be maintained at reduced temperature or otherwise maintained in an unformulated state, and prepared only immediately prior to the actual administration. In the instance where critical care is concerned and emergency room procedures may be involved, the need for formulation of the dosage form can heighten the risks associated with the procedure by the delay of its commencement. Alternately, the dosage forms may be prepared and maintained in refrigeration, however, such dosage forms maintained under reduced temperature or refrigeration must be brought to room temperature before administration, as they are otherwise not effective.

Accordingly, a need exists for the development of a dosage form and composition for the administration of an anesthetic, which achieves both improved shelf life and which can be maintained without refrigeration and can thereby be instantaneously able to be effective on administration to the patient. It is toward the achievement of the aforementioned objectives that the present invention is directed.

### SUMMARY OF INVENTION

In accordance with the present invention a composition and corresponding formulation are disclosed for an improved anesthetic dosage form. Correspondingly, such dosage form comprises a shelf stable composition for admixture on use. The composition comprises a quantity of hyaluronidase in a shelf stable form, and for example, in a first compartment, and a general anesthetic composition such as lidocaine or the like, either alone or in admixture with other adjuvants or additives, in a second compartment. Both compartments are preferably sealed, and on administration, the compartments are ruptured for intermixture of the components of the composition, followed by administration, such as by injection.

In a particular embodiment of the invention, the composition and formulation aforementioned is disposed within a multiply chambered syringe or like device, which effects the intermixing of the segregated components of the composition, and thereafter facilitates the administration of the resulting solution as by injection through a needle or the like, for delivery to the patient. The hyaluronidase is prepared in a shelf-stable form, as by reduction to a dry state. Such preparation may be accomplished, for example, by the lyophilization of the liquid form, and its reduction to a powder or a granular state. The other component of the composition may be disposed in a liquid form that is amenable to the rapid formation of a solution with the hyaluronidase when the components are brought together.

The composition, formulation and device of the invention is likewise embodied in a kit, where, for example, a suitable unit dosage form such as a multiply chambered syringe may be pre-manufactured or stored at room temperature for instantaneous use.

The formulations of the present invention have demonstrated remarkably improved shelf life and shelf stability, and require no refrigeration prior to use. Moreover, the presence of hyaluronidase in the said anesthetic composition is believed to accelerate and enhance the onset of the anesthetic state and thereby improves the quality of desensitization and corresponding commencement of treatment to the patient.

In a preferred embodiment of the present invention, the dosage form is disposed in a mixing chamber dispenser such as a plural chamber syringe, of which several are presently commercially available. The dosage forms may vary in volume and concentration of the ingredients, with 5 cc and 10 cc syringes being exemplary.

Accordingly, it is a principal object of the present invention to prepare a formulation and composition for the administration of an anesthetic that achieves an acceleration and rapid onset of the anesthetic state.

It is a further object of the present invention to provide a formulation and composition as aforesaid, that demonstrates improved shelf stability prior to use.

It is a still further object of the present invention to provide a composition and formulation as aforesaid that is capable of improved shelf life without the need for refrigeration.

It is a yet further object of the present invention to provide a unit dosage form and corresponding kit including the formulation and composition of the present invention disposed within separable containers within an administration device such as a syringe.

Other objects and advantages will become apparent to those skilled in the art from a review of the ensuing detailed description.

### DETAILED DESCRIPTION OF THE INVENTION

In accordance with the present invention, the foregoing objects and advantages are readily attained.

In its broadest aspect, the present invention relates to a composition and a formulation for an improved anesthetic. More particularly, the composition of the present invention features the formulation of an anesthetic agent with an ingredient that enhances its rate and scope of delivery and corresponding effect. In a particular aspect, the resulting composition exhibits an unexpected increase in its effective time, over known anesthetic compositions.

The ingredient that is believed to enhance and extend the longevity and effect of the present anesthetic composition is hyaluronidase, which is derived from a group of enzymes that are known to degrade certain tissue polysaccharides, known as glycosaminoglycans. Certain hyaluronidases are non-specific in their activity, and cleave hyaluronic acid, chondroitin and related polysaccharides, while other hyaluronidases are specific to hyaluronic acid. In turn, hyaluronic acid is a polysaccharide widely found in the extracellular connective tissue of animals, and is considered to function to bind cells together. Hyaluronidase has been previously identified as a spreading agent and has been used in treatments for glaucoma and the like, due to its ability to break down the vitreous humor. Interestingly, hyaluronidase has been used to assist in the promotion of withdrawal from anesthesia, in combination with an alpha adrenergic receptor antagonist (see U.S. Pat. No. 6,432,401). While such function is of particular and specific therapeutic importance, it does not suggest the valuable role for hyaluronidase that has been identified herein, and in fact teaches away from the same.

Specifically, the compositions of the present invention represent a synergistic combination of hyaluronidase and an anesthetic formulation, such as lidocaine, procaine, and the like, which achieves an unexpected enhancement in the delivery and onset of anesthesia. More particularly, the compositions of the invention may be formulated for unexpectedly improved shelf life and ease of administration, by preparation in a multi-component unit dosage form.

Accordingly, the invention extends to a unit dosage form for administration by a syringe or the like, which comprises a first hyaluronidase component, and a second anesthetic component, which are maintained in separation from each other prior to use and administration. The first component of hyaluronidase may be prepared in a solid or dry powder form and disposed in a fluid-impervious chamber or container. The preparation of hyaluronidase in powder form may proceed by freeze-drying (lyophilization) of the liquid substance, and its conversion into a powder by known techniques, such as prilling and the like. The powder preparation thus prepared is advantageously packaged and can be stably maintained and stored at room temperature prior to use, without exhibiting degradation or attenuation. In a particular embodiment, the unit dosage form may be a single unit dosage form, so that the spent dispenser, container, etc. may be discarded after use.

Suitable anesthetics that may be used for the preparation of the second component, are already well known and in longstanding use and circulation, and include by way of non-limiting examples, local anesthetics such as lidocaine, marcaine, polocaine, xylocaine, novocaine, procaine, prilocaine, bupivacaine, mepivacaine, carbocaine, etidocaine and chincocaine. The compositions of the invention may be formulated as anesthetic blocks, in the manner well known for such preparations.

In a particular embodiment, the anesthetic component comprises a mixture of lidocaine with a variety of like ingredients. Accordingly, the anesthetic component may coprise a mixture of lidocaine and an additional anesthetic selected from mepivacaine and bupivicaine.

In a further particular embodiment, the anesthetic component comprises lidocaine alone. In a further particular embodiment, the present composition may be prepared in a solution having a concentration ranging from 1.0% to about 5.0% by weight of active ingredient.

As stated above and in accordance with an important aspect of the invention, the present anesthetic compositions are formulated as separate components that are mixed on administration. The present invention therefore includes as an embodiment thereof, a kit for the admixture and conjoint administration of the anesthetic composition. Such kit may be prepared as, or for use with, a plural chamber syringe, where the anesthetic formulation is maintained in a solution that is separated from the hyaluronidase component by a fluid-tight barrier. In turn, the hyaluronidase, for example, in powdered form, is held in a sealed chamber and is only mixed on the activation of the syringe at the commencement of administration of the composition.

A suitable syringe device that can serve in the present invention, is disclosed by way of non-limiting example, in U.S. Pat. No. 6,817,987 to Vetter et al., the operative disclosure of which is incorporated herein by reference in its entirety. In the patent, the components of the administered composition are formulated and stored in fluid-tight separation and are only mixed on use, upon the insertion and depression of the plunger to force the piston within the device to rupture the barrier between the chambers and to effect the intimate mixture of the components of the composition prior to injection.

In a further aspect of the invention, the compositions that may be prepared and administered hereby may include other ingredients, such as complementary therapeutic agents, medicaments and the like, for release and treatment of the tissues at the site of injection. The choice and inclusion of such agents may vary within the skill of the art and could be determined by a skilled physician or veterinarian.

### EXAMPLES

The present invention will be better understood from a consideration of the following illustrative examples, wherein all percentages of ingredients are intended to be percent by weight.

### Example I

A first formulation comprises the mixture of approximately 20 cc of 2% Lidocaine with 1/100,000 epinephrine (stock solution), and with approximately 2000 units of Hyaluronidase (which is delivered in about 8-10 cc of volume with its dilutent). To this mixture is added 4% solution of plain Lidocaine, to fill a container or dispenser with a volume of 4% plain Lidocaine to give a total of 36 cc. The mixture thus prepared yields about 66 cc of total cocktail containing approximately 30 units of hyaluronidase per cc of 3% lidocaine and 1/400,000 epinephrine. A block uses 4 cc per patient of this mixture.

### Example II

In an alternate preparation, approximately 0.5 cc of hyaluronidase solution containing 100-110 units of drug with about 1.5 cc's of stock lidocaine/epinephrine 1/100,000 is prepared, and the resulting mixture is then supplemented with 2 cc of a 4% solution of Lidocaine alone, to yield a single 4 cc injection block.

### Example III

In a further formulation, a mixture of bupivicaine/lidocaine is prepared. Specifically, 2 cc's of the formulation prepared in accordance with Example I is mixed with 2 cc's of 7.5% mepivacaine which contains 50 units of hyaluronidase, and the resultant formulation is ready for administration.

The compositions and dosage forms of the invention are useful for the administration of anesthesia for a variety of therapeutic purposes and procedures. Thus, for example, the compositions may be prepared for administration as blocks in advance of various surgical procedures, and for the treatment or prevention of dental pain and ocular pain, whether in advance of a surgical procedure or in treatment of a pre-existing condition; and more generally, for pain management, e.g. as part of a treatment regimen

Various publications in addition to the immediately foregoing are cited herein, the disclosures of which are incorporated by reference in their entireties. The citation of any reference herein should not be deemed as an admission that such reference is available as prior art to the instant invention.

While the invention has been described and illustrated herein by references to the specific embodiments, various specific materials, procedures and examples, it is understood that the invention is not restricted to the particular material combinations of material, and procedures selected for that purpose. Indeed, various modifications of the invention in addition to those described herein will become apparent to those skilled in the art from the foregoing description, and such modifications are intended to fall within the scope of the present invention.

What is claimed is:

1. An anesthetic composition having improved efficacy and room temperature shelf-stability comprising a first component serving as an adjuvant, and a second component comprising an anesthetic formulation,
said first and said second components being segregated from contact with each other until use,
said first component comprising a shelf stable preparation of hyaluronidase, and said second component comprising an anesthetic selected from the group consisting of lidocaine, marcaine, polocaine, xylocaine, novocaine, procaine, prilocaine, bupivacaine, mepivacaine, carbocaine, etidocaine and chincocaine.

2. A composition according to claim 1 wherein said components are mixed at the time of administration.

3. A composition according to claim 1 wherein said hyaluronidase in prepared in a powdered form.

4. A composition according to claim 3 wherein said hyaluronidase is prepared in a powdered form by lyophilization.

5. A composition according to claim 1 wherein said anesthetic comprises a mixture of lidocaine and marcaine.

6. A composition according to claim 1 wherein said anesthetic comprises a mixture of lidocaine and epinephrine.

7. A composition according to claim 1 wherein said anesthetic comprises a mixture of lidocaine and bupivicaine.

8. A composition according to claim 1 wherein said anesthetic comprises lidocaine.

9. A composition according to claim 8 wherein said anesthetic is prepared in a solution having a concentration ranging from 1.0% to about 5.0% by weight of active ingredient.

10. A unit dosage form for the administration of an anesthetic by injection, comprising the composition of claim 1 prepared in a quantity of 4 cc for use as an anesthetic block.

11. The unit dosage form of claim 10 wherein said composition is disposed within a syringe having plural cavities each containing one of said components, whereby the operation of the syringe to dispense said composition will cause the mixing of the said components prior to dispensing, as by injection.

12. The unit dosage form of claim 10 prepared as a single dosage form.

13. A method for the administration of the anesthetic composition of claim 1 comprising the preparation of a dispensing device with a unit dosage form according to claim 11, and the operation of said syringe to dispense said composition at the intended site for anesthesia.
